SEQUENCE LISTING (28 sequences)

SEQ ID NO: 1 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 2 - protein, 155 aa, Homo sapiens [sequence omitted]

SEQ ID NO: 3 - protein, 288 aa, Homo sapiens; identical to SEQ ID NO: 1

SEQ ID NO: 4 - protein, 210 aa, Homo sapiens
MGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 5 - protein, 155 aa, Homo sapiens [sequence omitted]

SEQ ID NO: 6 - protein, 196 aa, Homo sapiens
MGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 7 - protein, 156 aa, Lycopersicon esculentum
AHLEYVENLNVKEQLVAGTLYYITLVATDAGKKKIYETKIWVKEWEDFKKVVEFKLVGDDSPNPGGITNVPFPNLPQFKDLARFAVQDYNKKENAHLEFVENLNVKEQVVAGIIYYITLVATDAGKKKIYETKILVKGWENFKEVQEFKLVGDATK

SEQ ID NO: 8 - protein, 158 aa, Gallus gallus
MAAGAAGSITTLPALPDDGGGGAFPPGHFKDPKRLYCKNGGFFLRINPDGRVDGVREKSDPHIKLQLQAEERGVVSIKGVSANRFLAMKEDGRLLALKCATEECFFFERLESNNYNTYRSRKYSDWYVALKRTGQYKPGPKTGPGQKAILFLPMSAKS

SEQ ID NO: 9 - protein, 171 aa, Homo sapiens
MHHHHHHHHHHHHDDDDKAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 10 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 11 - protein, 114 aa, Gallus gallus
DLDTDYCFGPGTDEKNCCVRPLYIDFRKDLQWKWIHEPKGYMANFCMGPCPYIWSADTQYTKVLALYNQHNPGASAAPCCVPQTLDPLPIIYYVGRNVRVEQLSNMVVRACKCS

SEQ ID NO: 12 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 13 - protein, 70 aa, Gallus gallus
GPETLCGAELVDALQFVCGDRGFYFSKPTGYGSSSRRLHHKGIVDECCFQSCDLRRLEMYCAPIKPPKSA

SEQ ID NO: 14 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 15 - protein, 179 aa, Bos taurus
MGITAGKSVLVLLAFLAFASCCYAAYRPSETLCGGELVDTLQFVCGDRGFYFSRPSSRINRRSRGIVEECCFRSCDLALLETYCATPAKSERDVSASTTVLPDDVTAYPVGKFFQYDIWKQSTQRLRRGLPAFLRARRGRTLAKELEALREAKSHRPLIALPTQDPATHGGASSKASSD

SEQ ID NO: 16 - protein, 181 aa, Sus scrofa
MGIPMRKPLLVLLVFLALASCCYAAYRPSETLCGGELVDTLQFVCGDRGFYFSRPASRVNRRSRGIVEECCFRSCDLALLETYCATPAKSERDVSTPPTVLPDNFPRYPVGKFFRYDTWKQSAQRLRRGLPALLRARRGRTLAKELEAVREAKRHRPLTARPTRDPAAHGGASPEASGHRK

SEQ ID NO: 17 - protein, 187 aa, Gallus gallus
MCAARQILLLLLAFLAYALDSAAAYGTAETLCGGELVDTLQFVCGDRGFYFSRPVGRNNRRINRGIVEECCFRSCDLALLETYCAKSVKSERDLSATSLAGLPALNKESFQKPSHAKYSKYNVWQKKSSQRLQREVPGILRARRYRWQAEGLQAAEEARAMHRPLISLPSQRPPAPRASPEATGPQE

SEQ ID NO: 18 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 19 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 20 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 21 - DNA, 574 bp, Gallus gallus
  source 1..574: mol_type = genomic DNA
gaggctggacggccgcggcagggggcgagcccgcccggcgctggcggcggcggccggcgggggcccggggcggcggggagccgccggggcccggcgcatggcggcgggggcggcggggagcatcaccacgctgccggcgctgcccgacgacgggggcggcggcgcttttccccccgggcacttcaaggaccccaagcggctctactgcaagaacggcggcttcttcctgcgcatcaaccccgacggcagggtggacggcgtccgcgagaagagcgatccgcacatcaaactgcagcttcaagcagaagaaagaggagtagtatcaatcaaaggcgtaagtgcaaaccgctttctggctatgaaggaggatggcagattgctggcactgaaatgtgcaacagaggaatgtttctttttcgagcgcttggaatctaataactataacacttaccggtcacggaagtactctgattggtatgtggcactgaaaaggactggacagtacaagcccggaccaaaaactggacctggacagaaagctatcctttttcttccaatgtctgctaaaagctga

SEQ ID NO: 22 - DNA, 474 bp, Lycopersicon esculentum
  source 1..474: mol_type = genomic DNA
atggcgcatctggaatatgtggaaaacctgaacgtgaaagaacagctggtggcgggcaccctgtattatattaccctggtggcgaccgatgcgggcaaaaaaaaaatttatgaaaccaaaatttgggtgaaagaatgggaagattttaaaaaagtggtggaatttaaactggtgggcgatgatagcccgaacccgggcggcattaccaacgtgccgtttccgaacctgccgcagtttaaagatctggcgcgctttgcggtgcaggattataacaaaaaagaaaacgcgcatctggaatttgtggaaaacctgaacgtgaaagaacaggtggtggcgggcattatttattatattaccctggtggcgaccgatgcgggcaaaaaaaaaatttatgaaaccaaaattctggtgaaaggctgggaaaactttaaagaagtgcaggaatttaaactggtgggcgatgcgaccaaatag

SEQ ID NO: 23 - DNA, 867 bp, Homo sapiens
  source 1..867: mol_type = genomic DNA
ctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 24 - DNA, 324 bp, Homo sapiens
  source 1..324: mol_type = genomic DNA
cgcgtggatggcgtgcgcgaaaaaagcgatccgcatattaaactgcagctgcaggcggaagaacgcggcgtggtgagcattaaaggcgtgtgcgcgaaccgctatctggcgatgaaagaagatggccgcctgctggcgagcaaatgcgtgaccgatgaatgctttttttttgaacgcctggaaagcaacaactataacacctatcgcagccgcaaatataccagctggtatgtggcgctgaaacgcaccggccagtataaactgggcagcaaaaccggcccgggccagaaagcgattctgtttctgccgatgagcgcgaaaagc

SEQ ID NO: 25 - DNA, 867 bp, Homo sapiens; identical to SEQ ID NO: 23
  source 1..867: mol_type = genomic DNA

SEQ ID NO: 26 - DNA, 468 bp, Homo sapiens
  source 1..468: mol_type = genomic DNA
atggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 27 - DNA, 528 bp, Homo sapiens
  source 1..528: mol_type = genomic DNA
ctcgagatgcatcatcatcatcatcatcatcatcatcatcatcatgatgatgatgataaagcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaacgcgt

SEQ ID NO: 28 - DNA, 324 bp, Homo sapiens; identical to SEQ ID NO: 24
  source 1..324: mol_type = genomic DNA

(19) United States
(12) Patent Application Publication
(10) Pub. No.: US 2024/0132903 A1
(43) Pub. Date: Apr. 25, 2024

(54) PLANT-BASED SYNTHESIS PRODUCTS

(71) Applicant: TIAMAT SCIENCES, Etterbeek (BE)

(72) Inventor: France-Emmanuelle ADIL, Etterbeek (BE)

(21) Appl. No.: 18/344,933

(22) Filed: Jun. 29, 2023

Related U.S. Application Data
(63) Continuation of application No. PCT/EP2022/050091, filed on Jan. 4, 2022.
(60) Provisional application No. 63/133,591, filed on Jan. 4, 2021.

(51) Int. Cl. C12N 15/82; A01H 6/82; C07K 1/22; C07K 14/485; C07K 14/50; C07K 14/65

(52) U.S. Cl. CPC C12N 15/8257; A01H 6/823; C07K 1/22; C07K 14/485; C07K 14/503; C07K 14/65; C12N 15/8205

(57) ABSTRACT

The present disclosure provides composition, systems, devices and methods for plant-based production of non-plant proteins through the use of heterologous genes for the expression of said non-plant proteins. Non-plant proteins can include, but are not limited to, mammalian proteins, cytokines, or growth factors.

### CROSS-REFERENCE

This application is a continuation of International Patent Application No. PCT/EP2022/050091, filed Jan. 4, 2022, which claims the benefit of U.S. Provisional Application No. 63/133,591, filed Jan. 4, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jun. 29, 2023, is named 59766-701_301_SL.xml and is 33,306 bytes in size.

### BACKGROUND OF THE INVENTION

Recombinant protein manufacturing describes the science of producing proteins within another structure other than their original organism. A gene of the protein of interest is inserted into a new organism with the ability of being multiplied. After this expression process, the recombinant proteins are extracted and purified. This technology has been implemented on a commercial scale since the early 1980s and is widely used today to produce a wide range of proteins from enzymes dedicated to the textile industry, to therapeutics, antibodies or growth factors commonly used in science.

Different expression systems have been developed over the years. Using yeast, bacteria, animal cells or human cells, they rely on similar production steps. Plant molecular farming is the green revolution of recombinant proteins manufacturing, offering an animal-free solution, efficient yield, high flexibility, and easy production scale-up path. Plant technology allows an exceptional flexibility with a production system costing 40 times less than our current cell-ag. competitors, and a scale-up potential meeting the requirements of the clean-meat sector. Plant molecular farming presents a solution to this problem. However, up to 80% of the production cost with plant molecular farming was linked to high particle burden of primary extracts, plant secondary metabolites, pigments and phenols. These additional clarification steps (extraction process) increase costs significantly. Thus, there is a need for more efficient systems for plant molecular farming.

### BRIEF SUMMARY

Provided herein are plant cells comprising a polynucleotide sequence encoding for a heterologous protease inhibitor gene or a functional variant thereof; and a polynucleotide sequence encoding for a mammalian gene or a functional variant thereof. Further provided herein are plant cells, wherein the polynucleotide sequence encoding for a heterologous protease inhibitor gene or a functional variant thereof and the polynucleotide sequence encoding for a mammalian gene or a functional variant thereof are within a bacterial or viral vector. Further provided herein are plant cells, wherein the bacterial vector is an Agrobacterium species. Further provided herein are plants cells, wherein the viral vector is Tobacco Mosaic Virus. Further provided herein are plants cells, wherein the mammalian gene is selected from the group consisting of TGF-β, IGF-1, IGF-2, human FGF-2, Activin A, BMP-4 and VEGF. Further provided herein are plants cells, wherein the heterologous protease inhibitor gene is SICYS8. Further provided herein are plants cells, wherein the polynucleotide sequence encoding for a heterologous protease inhibitor gene or a functional variant thereof and the polynucleotide sequence encoding for a mammalian gene or a functional variant thereof are RNA or DNA.

Provided herein are plant cells comprising a polynucleotide sequence encoding for a chicken FGF-2 gene or a functional variant thereof. Further provided herein are plants cells, wherein the polynucleotide sequence is within a bacterial or viral vector. Further provided herein are plant cells, wherein the polynucleotide sequence comprises a sequence with at least 75% sequence identity to SEQ ID NO: 21. Further provided herein are plant cells, wherein the functional variant comprises an insertion, deletion, or sequence variation compared to the full-length sequence. Further provided herein are plant cells, wherein the bacterial vector comprises an Agrobacterium species. Further provided herein are plant cells, wherein the viral vector is Tobacco Mosaic Virus. Further provided herein are plant cells, wherein the polynucleotide sequence is RNA or DNA.

Provided herein are plant cells comprising a polynucleotide sequence encoding for an IL-1(3 gene or a functional variant thereof. Further provided herein are plant cells, wherein the polynucleotide sequence is within a bacterial or viral vector. Further provided herein are plant cells, wherein the polynucleotide sequence comprises a sequence with at least 75% sequence identity to SEQ ID NO: 27. Further provided herein are plant cells, wherein the functional variant comprises an insertion, deletion, or sequence variation compared to the full-length sequence. Further provided herein are plant cells, wherein the bacterial vector comprises an Agrobacterium species. Further provided herein are plant cells, wherein the viral vector is Tobacco Mosaic Virus.

Provided herein are plant cells comprising a heterologous protease inhibitor protein or a functional variant thereof and a mammalian protein or a functional variant thereof. Further provided herein are plants cells further comprising a bacterial or viral vector. Further provided herein are plant cells, wherein the bacterial vector is an Agrobacterium species. Further provided herein are plant cells, wherein the viral vector is Tobacco Mosaic Virus. Further provided herein are plant cells, wherein the mammalian protein is from the group consisting of TGF-β, IGF-1, IGF-2, human FGF-2, Activin A, BMP-4 and VEGF.

Provided herein are plant cells comprising a chicken FGF-2 protein or a functional variant thereof. Further provided herein are plant cells, wherein the chicken FGF-2 protein comprises a sequence with at least 75% sequence identity to SEQ ID NO: 8. Further provided herein are plant cells, wherein the functional variant comprises an insertion, deletion, or sequence variation compared to the full-length sequence. Further provided herein are plant cells further comprising a bacterial of viral vector. Further provided herein are plant cells, wherein the bacterial vector comprises a Agrobacterium species. Further provided herein are plant cells, wherein the viral vector is Tobacco Mosaic Virus.

Provided herein are plant cells comprising an IL1-β protein or a functional variant thereof. Further provided herein are plant cells, wherein the IL1-β protein comprises a sequence with at least 75% sequence identity to SEQ ID NO: 9. Further provided herein are plant cells, wherein the functional variant comprises an insertion, deletion, or sequence variation compared to the full-length sequence. Further provided herein are plant cells, further comprising a bacterial of viral vector. Further provided herein are plant cells, wherein the bacterial vector comprises a Agrobacterium species. Further provide herein are plant cells, wherein the viral vector is Tobacco Mosaic Virus.

Provided herein are compositions comprising a mammalian protein or a functional variant thereof; and at least one of the following: flavonoids, rubisco, plant-derived alkaloids, cellulose, lignocellulose, legumalin, phaselin, 11S legumin type, 7s vicilin type, gliadin, zein, hordein, secalin, and glutenins. Further provided herein are compositions, wherein the mammalian protein is at least one of TGF-β, IGF-1, IGF-2, human FGF-2, Activin A, BMP-4 and VEGF. Further provided herein are compositions, wherein the at least one of the following comprises no more than 0.05% of the composition. Further provided herein are compositions, wherein the at least one of the following comprises no more than 0.03% of the composition. Further provided herein are compositions further comprising heparin. In some embodiments, heparin is present at no more than 2 μg/ml.

Provided herein are compositions, wherein the composition comprising a chicken FGF-2 protein or a functional variant thereof; and at least one of the following: flavonoids, rubisco, plant-derived alkaloids, cellulose, lignocellulose, legumalin, phaselin, 11S legumin type, 7s vicilin type, gliadin, zein, hordein, secalin, and glutenins. Further provided herein are compositions, wherein the at least one of the following comprises no more than 0.05% of the composition. Further provided herein are compositions, wherein the at least one of the following comprises no more than 0.03% of the composition. Further provided herein are compositions further comprising heparin. In some embodiments, heparin is present at no more than 2 μg/ml.

Provided herein are compositions comprising a human interleukin 1 beta (IL1-β) protein or a functional variant thereof and at least one of the following: flavonoids, rubisco, plant-derived alkaloids, cellulose, lignocellulose, legumalin, phaselin, 11S legumin type, 7s vicilin type, gliadin, zein, hordein, secalin, and glutenins. Further provided herein are compositions, wherein the at least one of the following comprises no more than 0.05% of the composition. Further provided herein are compositions, wherein the at least one of the following comprises no more than 0.03% of the composition. Further provided herein are compositions further comprising heparin. In some embodiments, heparin is present at no more than 2 μg/ml.

Provided herein are methods of manufacturing a mammalian protein comprising culturing the plant cells and extracting a mammalian protein encoded by the mammalian gene or the functional variant thereof to generate an extraction product. Further provided herein are methods, wherein the extraction product comprises the mammalian protein present in an amount of at least 40 μg per gram of biomass. Further provided herein are methods, wherein the mammalian protein is present in an amount of at least 45, 50, or 55 μg per gram of biomass. Further provided herein are methods, wherein the culturing comprises contacting a bacterial or viral vector to the plant cell using a syringe or spray method.

Provided herein are plant cells comprising a polynucleotide sequence encoding for a mammalian gene or a functional variant thereof, wherein the mammalian gene is selected from the group consisting of TGF-β, IGF-1, IGF-2, FGF-2, BMP-4, and VEGF, and wherein the plant cell is not derived from Oryza sativa. Further provided herein are plant cells, wherein the polynucleotide sequence encoding for a mammalian gene or a functional variant thereof are within a bacterial or viral vector. Further provided herein are plant cells, wherein the bacterial vector comprises an Agrobacterium species. Further provided herein are plant cells, wherein the viral vector comprises a Tobacco mosaic virus. Further provided herein are plant cells, wherein the polynucleotide sequence encoding for a mammalian gene or a functional variant thereof is RNA or DNA. Further provided herein are plant cells, wherein the functional variant comprises an insertion, a deletion, or a sequence variation compared to the mammalian gene.

Provided herein are plant cells comprising a mammalian protein or a functional variant thereof, and wherein the plant cell is not derived from Oryza sativa. Further provided herein are plant cells comprising a bacterial or viral vector. Further provided herein are plant cells, wherein the bacterial vector comprises an Agrobacterium species. Further provided herein are plant cells, wherein the viral vector comprises a Tobacco Mosaic Virus.

Provided herein are methods of manufacturing a mammalian protein comprising culturing the plant cells and extracting the mammalian protein or the functional variant thereof to generate an extraction product. Further provided herein are methods, wherein the extraction product comprises the mammalian protein present in an amount of at least at least 40 μg per gram of biomass. Further provided herein are methods, wherein the mammalian protein is present in an amount of at least 45, 50, 55 μg per gram of biomass. Further provided herein are methods, wherein the culturing comprises contacting a bacterial or viral vector to the plant cell using a syringe or spray method.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a general workflow for the expression of a mammalian protein, exemplifying a plant vector transforming plants into protein-expressing factories. The expressed proteins are extracted and purified to yield purified proteins derived from plants.

FIG. 2 shows a general workflow for a method of introducing heterologous nucleic acids into plants, exemplifying introduction of a bacterial vector or viral vector through agro-infiltration. A gene gun can be used to introduce nucleic acids into a plant without the use of a bacterial or viral vector.

FIG. 3 shows an exemplary plasmid construct used in Agrobacterium, showing a human FGF-2 CDS.

FIG. 4 shows a western blot of expressed human FGF-2 obtained from harvested infiltrated tobacco plants.

### DETAILED DESCRIPTION OF THE INVENTION

Provided herein are compositions, systems, devices and methods for plant-based production of non-plant proteins. As will be described in more detail herein are the use of (1) nucleic acid or polynucleotide sequences encoding for heterologous genes, (2) expression of heterologous proteins in plant, (3) large scale purification of such proteins (4) having stability and/or yield exceeding current commercially available options. In some embodiments, plant cells comprise a polynucleotide sequence encoding for a heterologous protease inhibitor gene and a polynucleotide sequence encoding a mammalian gene. In some embodiments, a composition comprises a mammalian protein, chicken FGF-2 or IL1-β; and at least one of the following: a flavonoid, rubisco, a plant-derived alkaloid, cellulose, lignocellulose, legumalin, phaselin, 11S ledumin type, 7s vicilin type, gliadin, zein, hordein, secalin, and glutenin. In some embodiments, a method of manufacturing mammalian protein comprises extracting the mammalian protein from a plant cell.

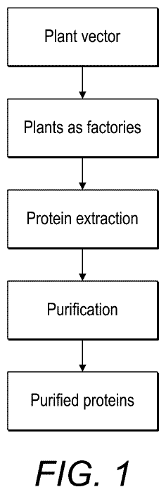

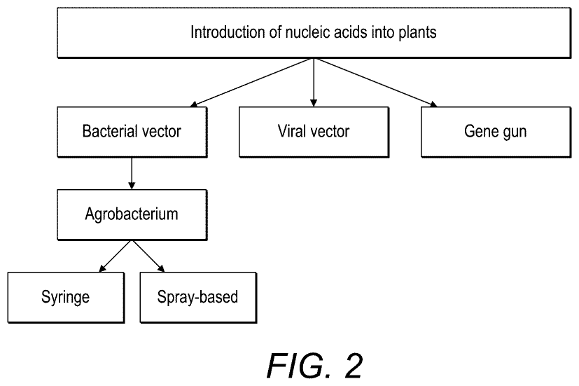

Expression of non-plant proteins can be achieved through the construction of a bacterial or viral vector comprising a sequence encoding for the non-plant protein. The bacterial or viral vector is introduced into the plant, thereby producing a plant capable of producing non-plant proteins. The non-plant proteins will be extracted and purified to yield a purified non-plant protein. As exemplified in FIG. 1, a plant vector transforms plants into protein-expressing factories. The expressed proteins are extracted and purified to yield purified proteins derived from plants. FIG. 2 describes methods that can be used to introduce nucleic acids into a plant.

As used herein, the term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Heterologous Nucleic Acid Transfer

Heterologous protein expression can be performed through the introduction of polynucleotide sequences encoding for heterologous proteins into the leaves of a plant, such as N. benthamiana. Introduction of the polynucleotide sequence into the plant with a vector can yield a plant capable of expressing heterologous proteins. Subsequent downstream processing and purification yields a purified protein. In some embodiments, the polynucleotide sequence can be DNA. In some embodiments, the polynucleotide sequence can be RNA.

An isolated and purified polynucleotide segment can be combined with transcription regulatory sequences using standard molecular biology methods to yield an expression cassette. Typically, these plasmids are constructed to provide for multiple cloning sites having specificity for different restriction enzymes downstream from the promoter. The isolated and purified DNA segment can be subcloned downstream from the promoter using restriction enzymes to ensure that the DNA is inserted in proper orientation with respect to the promoter so that the DNA can be expressed. Once the isolated and purified DNA segment is operably linked to a promoter, the expression cassette so formed can be subcloned into a plasmid or other vectors.

Provided herein are polynucleotide sequences for transfer into plant cells. In some embodiments, a polynucleotide sequence encoding the heterologous protein comprises a mammalian protein or a functional variant thereof. In some embodiments, the functional variant comprises an insertion, deletion, or sequence variation compared to the full-length sequence. In some embodiments, the polynucleotide sequence encoding the heterologous protein is a non-host protein. In some embodiments the mammalian protein is TGF-β, IGF-1, IGF-2, human FGF-2, activin A, BMP-4, VEGF, or chicken FGF-2. In some embodiments, the polynucleotide sequence encoding human FGF-2 comprises a sequence with at least 75% sequence identity to SEQ ID NOs: 23, 24, 25, 26, or 28. In some embodiments, the polynucleotide sequence encoding chicken FGF-2 comprises a sequence with at least 75% sequence identity to SEQ ID NO: 21. In some embodiments, the polynucleotide sequence encoding IL1-β comprises a sequence with at least 75% sequence identity to SEQ ID NO: 27.

In some embodiments, the polynucleotide sequence encoding human FGF-2 comprises a sequence with at least 75% sequence similarity to SEQ ID NOs: 23, 24, 25, 26, or 28. In some embodiments, the polynucleotide sequence encoding chicken FGF-2 comprises a sequence with at least 75% sequence similarity to SEQ ID NO: 21. In some embodiments, the polynucleotide sequence encoding IL1-β comprises a sequence with at least 75% sequence similarity to SEQ ID NO: 27.

Provided herein are plant cells comprising at least 1 polynucleotide sequence encoding a mammalian gene or functional variant thereof. In some embodiments, the functional variant comprises an insertion, deletion, or sequence variation compared to the full-length sequence. In some embodiments, the plant cell comprises a least 1 polynucleotide sequence encoding at least 1, 2, 3, 4, 5, or 6 mammalian, non-plant genes, or any functional variant thereof. In some embodiments, the plant cell comprises a least 1 polynucleotide sequence encoding at least 1-6 mammalian, non-plant genes or any functional variant thereof.

Provided herein is a plant cell comprising a polynucleotide sequence encoding a protease inhibitor to prevent endogenous degradation of the non-plant protein. In some embodiments, the protease inhibitor is a gene encoding a plant protease inhibitor or a non-plant protease inhibitor. In some embodiments, the protease inhibitor has at least 75% sequence similarity to SEQ ID NO: 22. In some embodiments, the protease inhibitor is SICYS8.

Provided herein are methods of introduction of a heterologous polynucleotide sequence into a plant using a bacterial or viral vector. In some embodiments, the vector is introduced using agroinfiltration. In some embodiments, agroinfiltration is performed by a syringe-based method. In some embodiments, agroinfiltration is performed using a spray that disperses a solution containing the vector onto the surface of the plant, thereby contacting the vector to the plant. In some embodiments, the solution contains a wetting agent. In some embodiments, the wetting agent comprises Tween 20.

### Expression of Heterologous Proteins

Provided herein are plants comprising any of the protein-encoding polynucleotides described above. The polynucleotides encode non-plant proteins, which can be expressed in the plant. In some embodiments, the non-plant proteins are mammalian proteins. In some embodiments the mammalian protein is TGF-β, IGF-1, IGF-2, human FGF-2, activin A, BMP-4, VEGF, or chicken FGF-2. In some embodiments, the plant cell comprises SICYS8 and/or said mammalian proteins, wherein the mammalian protein is selected from the group consisting of TGF-β, IGF-1, IGF-2, human FGF-2, IL1-β, activin A, BMP-4, VEGF, or chicken FGF-2. In some embodiments, human FGF-2 comprises a sequence with at least 75% sequence identity to SEQ ID NOs 1, 2, 3, 4, 5, or 6. In some embodiments, chicken FGF-2 comprises a sequence with at least 75% sequence identity to SEQ ID NO: 8. In some embodiments, IL1-β comprises a sequence with at least 75% sequence identity to SEQ ID NO: 9. In some embodiments, human FGF-2 comprises a sequence with at least 75% sequence similarity to SEQ ID NOs 1, 2, 3, 4, 5, or 6. In some embodiments, chicken FGF-2 comprises a sequence with at least 75% sequence similarity to SEQ ID NO: 8. In some embodiments, IL1-β comprises a sequence with at least 75% sequence similarity to SEQ ID NO: 9.

Provided herein are protease inhibitors expressed by a plant. In some embodiments, the plant expresses a protease inhibitor. In some embodiments, the protease inhibitor has 75% sequence identity to SICYS8. In some embodiments, the protease inhibitor has 75% sequence similarity to SEQ ID NO: 7.

The plant cell can include more than one heterologous protein. In some embodiments, the plant cell comprises at least one protease inhibitor protein or any functional variant thereof and at least one non-plant or mammalian protein or any functional variant thereof. In some embodiments, the plant cell comprises SICYS8 and at least 1 non-plant or mammalian protein. In some embodiments, the plant cell comprises 1) SICYS8 and 2) human FGF-2, chicken FGF-2, IL1-β, or any combination thereof.

### Vectors

Provided herein are vectors for use in transfer of heterologous polynucleotides into plant cells. In some embodiments, the vector is bacterial or viral. Exemplary bacterial vectors can be Agrobacterium species. Exemplary viral vectors can be Tobacco mosaic virus (TMV). In some embodiments, the bacterial vector is Agrobacterium tumefaciens. In some embodiments, the viral vector is Tobacco mosaic virus (TMV), tobacco rattle virus (TRV), tobacco etch virus (TEV), cowpea mosaic virus (CPMV), potato X virus (PVX), or any variant or strain thereof.

Agrobacterium tumefaciens is a soil-borne pathogen that is widely used to introduce heterologous polynucleotides into plant cells, including plant cells from a plant. A. tumefaciens transfers a particular polynucleotide segment of a tumor-inducing (Ti) plasmid into the nucleus of infected host cells. Advantageously, heterologous polynucleotides can be placed between the borders of the Ti plasmid and transferred to plant cells.

A polynucleotide sequence of interest can be introduced into a competent bacterial strain for nucleic acid transfer (e.g., Agrobacterium) via conventional transformation methods. The bacterial strain can be used to introduce the nucleic acid of interest into a plant, plant part, tissue, or cell. Many vectors are available for transformation of Agrobacterium. These typically carry at least one T-DNA border sequence and can include vectors such as pCambia, pSim24 or any variant thereof.

Agrobacterium transformation can involve the transfer of a binary vector carrying the foreign nucleic acid of interest to an Agrobacterium strain which may depend on the complement of vir genes carried by the host Agrobacterium strain either on a co-resident Ti plasmid or chromosomally. The transfer of the recombinant binary vector to Agrobacterium can be accomplished by a tri-parental mating procedure using E. coli carrying the recombinant binary vector, a helper E. coli strain that carries a plasmid and which is able to mobilize the recombinant binary vector to the target Agrobacterium strain. Alternatively, the recombinant binary vector can be transferred to Agrobacterium by DNA transformation.

A polynucleotide of interest can be transformed into the Agrobacterium strain or other bacterial strain competent for nucleic acid transfer for subsequent transformation of a plant using the methods as disclosed herein. In some embodiments, the Agrobacterium is transformed using electroporation. In some embodiments, the nucleic acid is a polynucleotide construct comprising an expression cassette that comprises functional elements that allow for expression of a polynucleotide of interest in a plant following its introduction via the Agrobacterium-mediated transformation methods of the disclosure.

An expression cassette can comprise a nucleic acid encoding a polynucleotide that confers a property that can be used to detect, identify or select for transformed plant cells and tissues (e.g., a marker for the selection of transformed cells). The nucleic acid encoding the marker may be on the same expression cassette as the nucleotide sequence of interest, or may be co-transformed on a separate expression cassette. In some embodiments, the nucleic acid encoding the marker can be the nucleotide sequence of interest. Thus, the nucleic acid of interest comprises an expression cassette that further comprises a nucleotide sequence conferring resistance to a selection agent, and thus, selecting comprises culturing the Agrobacterium-inoculated a plant tissue or cell thereof in a medium comprising the selection agent, and selecting a transformed plant tissue or cell thereof comprising the nucleic acid of interest.

### Delivery of Nucleic Acids

Disclosed herein are steps, or the method of manufacturing, directed to introducing an isolated and purified DNA sequence, such as a polynucleotide sequence containing a heterologous protein (i.e. mammalian or non-plant protein), into a plant cell to produce a transformed plant cell. In some embodiments, the transformed plant cell exhibits transient expression of the heterologous protein.

Cells of the plant tissue source can be embryogenic cells or cell-lines that can regenerate fertile transgenic plants and/or seeds. The cells can be derived from either monocotyledons or dicotyledons. Suitable examples of plants include, but are not limited to, wheat (e.g., Triticum species), rice (e.g. Oryza species), Nicotiana (e.g., Nicotiana benthamiana), Arabidopsis, tobacco (Nicotiana species), maize (e.g., Zea species), soybean (e.g., Glycine species), oat (e.g., Avena), and the like.

The choice of plant tissue source for transformation can depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells.

The transformation is carried out under conditions directed to the plant tissue of choice. The plant cells or tissue are exposed to the DNA carrying the isolated and purified DNA sequences for a period of time. This may range from a few minutes to 2-15 days co-cultivation in the presence of plasmid-bearing Agrobacterium cells. Buffers and media used can vary with the plant tissue source and transformation protocol.

After transformation, the plant is grown for a period time in order to allow for accumulation of the heterologous protein within the plant. In some embodiments, the period of time is from about a 1 hour to about 15 days. In some embodiments, the period of time is 1 hour, 2 hours, 6 hours, 12 hours or 1, 5, 6, 7, 8, 9, 10, or 15 days. In some embodiments, the period of time is from 1-12 hours, 1 hour −1 day, 1-5 days, 1-10 days, 1-15 days, 1-5 days, 1-10 days, 1-15 days, 5-6 days, 5-7, days, 5-8 days, 5-9 days, 5-10 days, or 5-15 days.

The method of manufacturing a mammalian protein includes culturing a plant cell in a growth media. Introduction of a nucleic acid sequence into a plant cell provides the plant the ability to express a protein. Following expression of the protein, the mammalian protein is extracted to generate an extraction product. Growth media can include soil or agar-based media supplemented with factors necessary for growth or germination.

Introduction of a nucleic acid into a plant cell may involve use of a bacterial or viral vector. In some embodiments, the bacterial vector is an Agrobacterium species. In some embodiments, the bacterial vector is Agrobacterium tumefaciens. In some embodiments, the viral vector is Tobacco mosaic virus (TMV), tobacco rattle virus (TRV), tobacco etch virus (TEV), cowpea mosaic virus (CPMV), potato X virus (PVX), or any variant or strain thereof.

In some embodiments, the bacterial or viral vector is introduced to the plant cell by contacting the plant cell with the bacterial or viral vector. The contacting may involve mediating physical contact between the plant cell and the bacterial or viral vector. In some embodiments, the physical contact is mediated through use of a syringe-based system. In some embodiments, the contacting is performed on the leaves of a plant, thereby forming an infiltrated leaf.

Introduction of a nucleic acid can include dispersal of a solution to initiate contact of the nucleic acid into a plant cell. In some embodiments, introduction of a nucleic acid may include using a spray method. The spray method can comprise a solution containing bacterial or viral vector. The bacterial or viral vector can be suspended in a solution an sprayed using a spray-based nozzle or sprinkler system to disperse the solution into a mist that covers a broad area containing plants. In some embodiments, the introduction is performed by contacting a syringe containing the bacterial or viral vector to a plant cell.

Introduction of a polynucleotide sequence encoding a non-plant protein may use a method of introduction of a polynucleotide instead of using a vector-based system. In some embodiments, introduction of a polynucleotide sequences into a plant cell comprises use of a gene gun. In some embodiments, the polynucleotide sequence is RNA or DNA.

### Large Scale Purification

Purification of large amounts of protein can include purification of large amounts of plant biomass in order to recover substantial amounts of the non-plant or mammalian protein. This may include the use of multiple plants in a vertical farm or large greenhouse that provides a scalable and controlled environment to grow multiple plants that produce the protein of interest.

After culturing the plant cells, a mammalian protein accumulates in a plant cell and is extracted to generate an extraction product or composition comprising the mammalian protein. The composition may also comprise impurities or non-mammalian components. In some embodiments, the impurities may be present in trace quantities. In some embodiments, the impurities are flavonoids, rubisco protein, proteases, proteins, plant-derived alkaloids, cellulose, lignocellulose, legumalin, phaselins, 11S ledumin type, 7s vicilin type, gliadins, zeins, hordeins, secalins, or glutenins. During purification, the purified protein can contain trace amounts on plant-derived materials or impurities. The impurities may comprise a portion of the composition. In some embodiments, the impurities constitute no more than 0.1% of the composition. In some embodiments, the impurities constitute no more than 0.07%, 0.06%, 0.05%, 0.04%, 0.03%, 0.02%, or 0.01% of the composition. In some embodiments, the impurities constitute 0.0%-0.1% of the composition.

In some embodiments, the composition may further comprise an anticoagulant. In some embodiments, the anticoagulant is heparin or a salt thereof. In some embodiments, the anticoagulant is present at 0.1 μg/ml to 2 μg/ml. In some embodiments, the anticoagulant is present at no more than 2 μg/ml.

Purification of the mammalian protein can be performed by purification methods commonly known by one skilled in the art. In some embodiments, the mammalian protein is purified by affinity chromatography, ion-exchange chromatography, size-exclusion chromatography, immobilized metal affinity chromatography, or any combination thereof.

After purification, post-processing steps can be used to optimize the functionality of the purified protein. In some embodiments, an affinity tag is fused to a non-plant protein to allow for ease of purification using affinity chromatography or purification. The affinity tag can be removed to prevent interference of the tag with protein function. In some embodiments, a protease can be used to cleave an affinity tag used in affinity chromatography, thereby generating a “tag-less” non-plant protein. In some embodiments, the affinity tag can be a poly-His-tag, a Strep-tag, an E-tag, or other epitope tags commonly used in purification. In some embodiments, the protease is an enterokinase.

In some embodiments, the non-plant or mammalian protein is produced as a proprotein or as a zymogen, thus not functional without additional processing steps. In some embodiments, a protease is used to render the proprotein into a mature protein.

Extraction of the mammalian protein may exceed yields that are currently available through other methods. Extracting the mammalian protein to generate the extraction product. In some embodiments, the extraction product comprises a mammalian protein is present in an amount of at least 40 μg per gram of biomass. In some embodiments, the extraction product comprises a mammalian protein is present in an amount of at least 40, 41, 42, 43, 44, 45, 46, 47, 48, 49, 50, 51, 52, 53, 54, or 55 μg per gram of biomass. In some embodiments, the extraction product comprises a yield of 40-55 μg per gram of biomass.

### Method of Manufacture

Provided herein are methods of manufacturing a mammalian from a plant cell comprising the polynucleotide sequences encoding the non-plant protein. Plants containing the non-plant protein or mammalian protein can be cultured until a desired amount of protein is produced. The extraction of the plant generates an extraction product comprising a mammalian protein.

Culturing of plant cells can be performed by growing plants or plant cells in suitable growth media such as soil or agar supplemented with factors needed for growth or seed germination. In some embodiments, the culturing of plant cells or plants occurs in a green house, or other facility that provides a controlled environment that allows for plant growth. Culturing of plants in a scalable manner can provide an increase of potential biomass harvested in the same area used. In some embodiments, vertical farming techniques are used to grow the plants.

The method of manufacturing the mammalian protein may exceed yields that are currently available through other methods. Extracting the mammalian protein to generate the extraction product. In some embodiments, the extraction product comprises a mammalian protein is present in an amount of at least 40 μg per gram of biomass. In some embodiments, the extraction product comprises a mammalian protein is present in an amount of at least 40, 41, 42, 43, 44, 45, 46, 47, 48, 49, 50, 51, 52, 53, 54, or 55/is per gram of biomass. In some embodiments, the extraction product comprises a yield of 40-55/is per gram of biomass.

Following purification, the non-plant or mammalian protein can be further sterilized. In some embodiments, the sterilization comprises filtering, irradiation, endotoxin purification, or any combination thereof.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

### EXAMPLES

### Example 1: Germination of Nicotiana benthamiana Seedlings

To prepare Nicotiana benthamiana for introduction of a transgene, N. benthamiana seeds are incubated for 3 days on moist jiffy pads at 30 degree Celsius in an incubator with a 16-hour light and 8-hour dark cycles. Germinated seeds are transferred into moist soil for further growth.

### Example 2: Preparation of the Plant Expression Vector

Preparation of Competent E. coli

E. coli must be competent in order to take up an exogenous nucleic acid such as a plasmid. To do this, 100 μL of E. coli is added to 1 mL of liquid LB or SOC and are incubated overnight at 37° C., 225 RPM.

TSS buffer is prepared (30 mM MgCl₂, 5% DMSO, 10% PEG (3350 or 5000)) in LB medium under sterile conditions using aseptic technique and filtered under the laminar flow biochemical hood. The prepared buffer is stored at 4° C. until needed.

Following the overnight culture, a small amount of the overnight culture is sub-cultured into a larger volume of LB for 2-3 hours until the OD600 reaches 0.3-0.4. The culture is then centrifuged at 2700×g for 10 minutes at 4° C., the supernatant is removed, and the pelleted cells are resuspended in 5 ml of pre-chilled TSS buffer. The resuspended cells are chilled on ice for 15 minutes. After chilling, the chilled cells will be aliquoted into appropriate freezer tubes and frozen at −80° C.

### Preparation of the Bacterial Vector

1 μL of pCambia2300 (Marker Gene Technologies, PN: M1709) (plasmid (— 1000 pg) is added to 1004 of competent E. coli cells and is mixed well and incubated for 30 minutes at 4° C. 900 μL of liquid LB media supplemented with 20 mM glucose is added to the chilled bacteria and incubated at 37° C. at 225 RPM for 1 hour.

900 μL of the incubated bacteria is added to 40 mL of liquid LB media supplemented with 50 μg/mL kanamycin and incubated overnight at 37° C. at 225 RPM. The plasmid is purified according to the GenElute plasmid MidiPrep purification kit according to the manufacturer's instructions (Sigma, PLD35).

The remaining 100 μL is plated onto LB-agar supplemented with the appropriate antibiotic and stored at 4° C. Bacterial stocks are prepared for long-term storage by preparing a stock containing a final concentration of 25% glycerol.

### Example 3: Transformation of Agrobacterium Tumesfaciens

Agrobacterium tumefaciens LBA4404 (Invitrogen) is electroporated with the nucleic acid vector. Bacterial colonies are verified for uptake of the nucleic acid vector prior to electroporation into Agrobacterium.

Agrobacterium LBA4404 is thawed on ice. 1 μL of the vector is added to 204 of competent cells and mixed gently. Cell:plasmid mixture is added to a 0.1 cm electroporation cuvette (Bio-rad) on ice. The Gene Pulser II is configured with the following parameters: 25 g, 300 Ω, and 2-2.5 kV. 1 mL of SOC media is added to electroporated cells and transferred to a 1.5 ml tube and incubated for 1 hour at 28° C., shaking at 100 rpm. Plate 50-100 μl of cells on LB agar plates with 50 μg/ml kanamycin and 100 μg/ml streptomycin and incubate for up to 48 hours at 28° C.

### Example 4: Infiltration of N benthamiana via Syringe

A clonal population of Agrobacterium is grown in 5 ml of LB with kanamycin and streptomycin. 1 mL of the overnight culture is inoculated into 25 mL LB supplemented with 20 μM acetosyringone and grown overnight at 28° C. at 100 rpm. Plate 50-100 μl of cells on LB agar plates with 50 μg/ml kanamycin and 100 μg/ml streptomycin for up to 48 hours at 28° C.

After overnight culture, bacteria is precipitated at 5000 x g for 15 minutes and resuspended such that the ° Da) is equal to about 0.5 with MM media (10 mM MES, 10 mM MgCl₂, pH 5.6 (KOH). After adjustment, incubate the flask at room temperature for 1-3 hours or overnight.

Infiltration via syringe is performed by using a 5 ml syringe without a needle. Applying the syringe to the underside of the leaf while exerting counter pressure with your finger on the other side. Successful infiltration is overused as spreading (i.e. wetting) area in the leaf. Infiltration should be performed when fully expanded N. benthamiana leaves are present (preferentially in the morning when the stomata are open) with the A. tumefaciens suspension at different places to the abaxial part using a 5 ml syringe without needle.

After 2-5 days, expression of the transgene can be observed to verify successful infiltration.

### Example 5: Spray-Mediated Infiltration of N benthamiana

Infiltration of Agrobacterium can also be performed using a spray method (FIG. 2) that would be able to be scalable compared to syringe-based infiltration. From the back-up plate or bacteria glycerol stock, take cells and inoculate 5 ml of selective LB media (streptomycin/kanamycin). Incubate overnight at 28° C. with 225 rpm shaking. 5 ml of Agrobacterium culture is added to 250 ml of selective LB-media (streptomycin/kanamycin) and will be incubated for 48 hours to 72 hours at 28° C. with 225 rpm shaking. After incubation, the cells are centrifuged at 4000×g for 10 minutes. Discard the supernatant. Resuspend the cells in 10 ml of MM medium (10 mM MES, 10 mM MgCl₂, pH 5.6 (KOH)) and incubate at room temperature for one hour in the dark. MINI medium is added to adjust the OD at 1.3-1.5 in a total volume of 500 ml, add Tween 20 for 0.1% (v/v) final concentration. Spray the solution directly onto the plants in the incubator at 23° C. Leave the plant for protein expression for 10 to 14 days with daily watering.

### Example 6: Protein Extraction

For purification, plant material is extracted using the buffer containing 20 mM citric acid, 20 mM Na₂HPO₄, and 30 mM NaCl in a 5:1 (v/w) buffer: biomass ratio. The extraction is carried out at pH 4.

To prepare 50 ml of the extraction buffer, weigh 192 mg of acid citric, 120 mg of NaH₂PO₄, and 88 mg of NaCl. In a beaker, the reagents are diluted in 50 ml in distilled water, and the pH adjusted to pH 4. Store the extraction buffer at 4° C.

Ground leaf material supplemented with pre-chilled extraction buffer is incubated at room temperature under constant agitation for 30 min followed by centrifugation at 10,000×g for 15 min. The supernatant is filtered using Miracloth followed by incubation of the filtrate for 20 min at room temperature and centrifugation for 30 min at 10,000×g at room temperature.

For poly-histidine-tagged proteins, Ni-NTA Dynabeads are used. For preparation of protein prior to purification via Dynabeads, transfer 50 μL (2 mg) Dynabeads™ magnetic beads to a microcentrifuge tube and is placed on a magnet for 2 minutes. Aspirate and discard the supernatant. The sample (prepared in 1X Binding/Wash Buffer) is added to the beads and is incubated for 5 minutes at room temperature (or colder if the protein is unstable at room temperature). The incubation time may be increased up to 10 minutes. The tube is placed on the magnet for 2 minutes, then discard the supernatant. The beads are washed 4 times with 300 μL 1X Binding/Wash Buffer by placing the tube on a magnet for 2 minutes and discarding the supernatant. The beads are resuspended thoroughly between each washing step. To use bead/protein complexes in other applications, the bead/protein complex is resuspended in a suitable volume of 1X Pull-down Buffer (or other buffer compatible with your downstream application). 100 μL His-Elution Buffer is added to the suspension and is incubated on a roller for 5 minutes at room temperature (or colder if the protein is unstable at room temperature). A magnet is applied for 2 minutes and the supernatant containing the eluted histidine-tagged protein is transferred to a clean tube. To measure protein concentration, a Bradford assay or protein quantification using a Qubit fluorometer is used.

### Enterokinase Cleavage

Enterokinase storage buffer preparation 10 ml (20 mM Tris-HCl, 200 mM NaCl, 2 mM CaCl₂) and 50% glycerol)

Enterokinase Reaction:

The enterokinase cleavage reaction is prepared with the fusion protein mixed into a reaction mixture composed of the reaction buffer (200 mM Tris-HCl, 500 mM NaCl, 20 mM CaCl₂, pH 8) and the enterokinase enzyme. A negative control is generated (no enterokinase) in which 5 μl of Enzyme Storage buffer is used in place of Enterokinase. Collect all components by a brief centrifugation. The reaction is incubated at 25° C. and aliquots are removed for analysis at various timepoints, and prepare aliquots for analysis by SDS-PAGE. The optimal time for cleavage analysis is analyzed by comparing the amount of cleaved and uncleaved protein at each time point via SDS PAGE. Another Dynabeads purification is performed to remove any uncleaved protein as well as cleaved poly-His tag. Residual enterokinase is removed using the Enterokinase Removal Kit (Sigma Aldrich Cat No: PRKE). To measure protein concentration of the supernatant, a Bradford assay or protein quantification using a Qubit fluorometer is used.

### Example 7: Plant Cultivation Conditions

For this study, plants were cultivated in a greenhouse or a growth container.

### Greenhouse Cultivation

Plants were seeded into Proptek propagation 231 deep cell tray (1020 format) and filled with ProMix fine soil using a Speedy Seeder (Carolina Greenhouses, NC, USA). The conditions for germination and cultivation are described:

150+-50 μmol m⁻²s⁻¹ for the germination that takes place into the Percival chambers;

The temperature was set at 26° C. during the day and 25° C. during the night. Svenson clothes were always closed on the top. Supplemental lightning was given to the plants with High-Pressure Sodium (HPS) to complete the photoperiod, and light was provided once the external sensor of the greenhouse was below 150 micromoles first and then 200 micromoles; the photoperiod was set at 16 hours of light and 8 hours of darkness; the environmental humidity was set at 50% to reduce microbiological problems.

The seeds were irrigated with a nutrient solution obtained diluting a stock MiracleGro 24-8-16+micronutrients with the following characteristics: Electrical conductivity: 1 dS/m±0.2 and pH: 5.7±0.2.

The MiracleGro stock was prepared by diluting 200 g of fertilizer/liter of stock. The transplant takes place at 14 days after seeding (DAS 14). The transplant was performed manually from the seeded flat into 3.5 inch×3.5 inch square pots filled with MiracleGro Potting soil with wetting control agents. The pots were filled the day before the transplant and soaked with water to arrive at the field capacity. In order to provide an irrigation system using 1020 trays, two different 1020 flats were placed on top of each other: one with holes and one with no holes at the bottom. In the top, 1020 flats held 18 pots and, in this way, it was possible to fill and drain the pots all at once.

The greenhouse was set up with benches that were used only to facilitate the watering operations. Plants were spaced for the first time at DAS 21, placing only 9 plants/flat instead of 18.

At DAS 28, temperatures were lowered at 22° C.±2, plants were spaced at 5 or 6 per flat before agroinfection takes place. Plants were harvested after 4 more days of cultivation, the first two days a lower temperature (22±2° C.) was used. The last two days were cultivated with conditions adopted during cultivation (26±2° C. during the day and 25±2° C. at night).

### Growth Container Cultivation

The container cultivation conditions were provided: 150+-50 μmol m⁻²s⁻¹ (germination that takes into the germination racks with Jiffy 44 seeded, see below), temperature set at 26° C. during the day and 25° C. during the night; the photoperiod was set at 16 hours of light and 8 hours of darkness; and the environmental humidity was set at 70% during germination and then, for the latter phases, at 50% to reduce the risk of microbiological problems.

Plants were sown using the Speedy Seeder (Carolina Greenhouses, NC, USA) in a 1020 flat containing Jiffy 7 plugs prehydrated and contained into a plastic net. After the seeding operations, the Jiffys were spaced into QuickPot 45 R which can host 45 sown Jiffys. The Quickpots trays were placed onto the germination racks and after 2-3 days the germination should be evident. After 1 week, environmental humidity was decreased to 50%. After 2 weeks, plants were spaced by placing 5 plants/Quickpot tray and grown into the cultivation racks for another 2 weeks, where they will be agro infected with a solution containing Agrobacterium tumefaciens. The harvest took place 4 days later by removing the trays from the irrigation systems and manually cutting the leaves.

The seeds and plants were fertigated with a nutrient solution described below, which stock is composed the diluted fertilizer prepared as shown in Table 3.

TABLE 3
Recipe of Fertilizer used in Growth Chambers
Tank A

| | | | Volume | % of |
|---|---|---|---|---|
| | Mass | | to Add | Final |
| Component | (g) | ml/grams | (L) | Solution |
| Jack's B Calcium Nitrate | 3900 | 0.45 | 1.76 | 8.8 |
| Iron EDDHA Chelate 6% | 200 | 0.52 | 0.1 | 0.5 |
| Water | | | 18.14 | 90.7 |
| Total Tank A Fertilizer Volume | | | 20 | 100 |

Tank B

| | | | Volume | % of |
|---|---|---|---|---|
| | | | to Add | Final |
| Component | Mass | ml/grams | (L) | Solution |
| Jack's 5-12-26 Part A | 3000 | 0.47 | 1.41 | 7.05 |
| Monopotassium phosphate | 700 | 0.47 | 0.33 | 1.65 |
| Water volume | | | 18.26 | 91.3 |
| Total Tank Fertilizer B Volume | | | 20 | 100 |

The stock was further prepared with the following characteristics: Electrical conductivity: 1 dS/m+−0.2 during the first week after transplant, then for the latter growing phases 1.5 dS/m+−0.2, and pH: 5.7+-0.2.

### Example 8: Expression of human FGF-2 in N benthemiana via Agrobacterium transformation

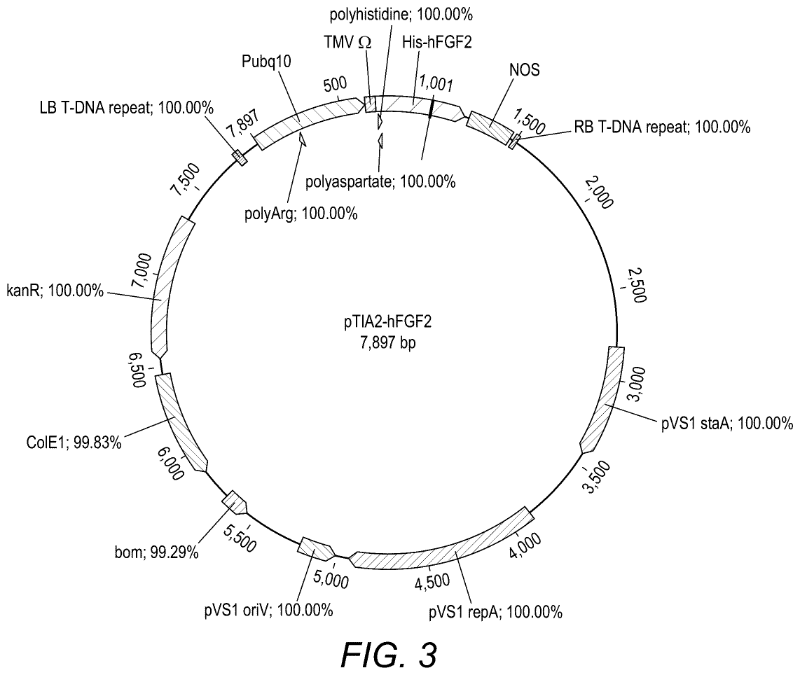

Plasmid construct pTIA2-hFGF2 was synthesized by Genscript using the backbone of pCAMBIA0380 (FIG. 3). The plasmid construct includes a ubiquitin-10 promoter from Arabidopsis thaliana in order to drive expression of the TMV Omega enhancer sequence followed by N. benthemiana (tobacco plant) codon optimized human FGF-2 (SEQ ID NO: 24) with a 6x Histidine tag on the N′ terminal end of the hFGF-2 polypeptide. The CDS is followed by the NOS terminator. No plant-specific selectable marker was included.

Agrobacterium hFGF-2 Stock Preparation:

The plasmid construct pTIA2-hFGF2 was electroporated into Agrobacterium tumefaciens strain GV3101 (AbCam) as described in Example 3 (25 g, 300 Ω, and 2-2.5 kV). The electroporated Agrobacterium was grown for 2 days at 28° C. on LB agar plates supplemented with 200 mM acetosyringone, 25 mg/L rifampicin, 50 mg/L gentamicin, and 100 mg/L kanamycin.

A single colony was selected and used to prepare a 25% glycerol stock and be frozen at −80C. The presence of the hFGF-2 was confirmed by PCR.

Agrobacterium preparation for N. benthemiana infiltration

Frozen glycerol stocks of transformed Agrobacterium containing the hFGF-2 expressing plasmid were streaked on LB agar plates supplemented with 200 mM acetosyringone, 25 mg/L rifampicin, 50 mg/L gentamicin, and 100 mg/L kanamycin and incubated at 28° C. for 2-3 days.

After incubation, a single colony was selected and used to inoculate a 500 ml baffled flask containing 100 mL of LB broth supplemented with 200 mM acetosyringone, 25 mg/L rifampicin, 50 mg/L gentamicin, and 100 mg/L kanamycin. The culture was incubated in a shaking incubator for 16-18 hours at 220 RPM.

After incubation, 100 mL of the bacterial culture was transferred to two 50 mL falcon tubes. The falcon tubes were centrifuged at 4000 RPM for 30 minutes. The supernatant was discarded.

1L of MMA medium containing 10 ml 1M IVIES at pH5.6, 10 ml of 1M MgCl₂, 1 ml of 200 uM acetosyringone was prepared and the bacterial pellet was resuspended in the 1L of MMA medium. The resuspended bacteria was incubated for 2 hours at 22° C.

Following incubation, 10 μM Lipoic acid, 100 mg/L L-cysteine, 125 mg/L STS, 75 mg/L DTT, 0.002% Pluronic F-68 was added to the resuspended culture. Silwet L-77 was added to attain a concentration of 0.01%.

4 week old tobacco plants in a vacuum desiccator were vacuum infiltrated by applying 0.02 mPa for 1 minute.

Incubate N. benthemiana plants were vacuum infiltrated in a growth chamber at 22° C. for two days and then 24° C. for a third day.

### Example 9: Extraction of Total Soluble Protein (TSP) from Tobacco Leaves

An extraction buffer was prepared by mixing a solution containing the following concentrations of reagents: 50 mM sodium phosphate pH 7.4, 300 mM NaCl, 10 mM imidazole, 0.1% TritonX-100, 40 mM ascorbic acid, EDTA-free protease inhibitor cocktail tablets (as per manufacturer instruction).

The leaves of the infiltrated (3-days post-infiltration) N. benthemiana plants from Example 8 were harvested and frozen at −80° C.

3 grams of the frozen leaf tissue was placed into a 50 mL SPEX tube with two 11 mm metal beads and 10 mL of extraction buffer on ice. The tubes were placed in tube adaptors for the SPEX Geno/Grinder. The adaptors are prechilled at −80° C.

The frozen leaves were ground in the SPEX grinder using five 30 seconds bursts at 1500 RPM with 30 seconds of rest time between bursts.

Following the grinding, the tubes were centrifuged at 4000 RPM for 10 minutes at 4° C. The pellet contains cellular debris. The leaf material and supernatant was poured through 4 layers cheesecloth into a new falcon tube and centrifuged at 7000×g for 30 minutes at 4° C. The supernatant containing the total soluble protein (TSP) was transferred to a new falcon tube.

The pH of the TSP was adjusted to a final pH of between 7 and 8 with KOH (potassium hydroxide). The pH adjusted TSP was centrifuged for 15 minutes at 7000 x g at 4° C. The supernatant was transferred to a new falcon tube.

### Example 10: Detection of Human FGF-2

### Protein Gel:

A BioRad gel rig loaded with a TGX (Tris Glycine Extended) 4-15% gradient gel prepared with TGS (25 mM Tris, 192 mM Glycine, 0.1% SDS, pH 8.6) running buffer.

2.5 μL of 4x Laemmli buffer and 7.5 μL TSP sample was prepared. A positive control was prepared with 10 ng of hFGF2. The samples were denatured by heating the samples at 70° C. for 10 minutes. 10 μL of sample was loaded into the wells and 5 μL of Pageruler prestained ladder (Thermofisher) in at least one well. The gel was run at 200V for 30 minutes.

### Immunoblot:

The BioRad Trans-Blot Turbo and Trans-Blot® Turbo™ Mini PVDF Transfer Packs were used to transfer protein from gel to membrane. The gel was transferred to an methanol activate PVDF membrane using the preset 3 minute TGX mini gel transfer protocol. The transferred membrane was place into a black box containing freshly made 0.75 grams bovine serum albumin and 25 mL of TBS-T blocking buffer and incubated with gentle shaking for 1 hour at room temperature or overnight at 4° C.

The blocking buffer was removed. 10 μL of monoclonal mouse anti-hFGF2 primary antibody (Invitrogen) with 25 mL TBST (1:2500 dilution) was added to the membrane and incubated with gentle rocking for either for 4 hours at room temperature or overnight at 4° C.

The primary antibody was removed and washed 3 times with TBST, where each was performed for 10 minutes at room temperature using 50 mL TBS-T.

The secondary antibody solution was prepared using anti-mouse NIR800 at 1:10000 by mixing 3 μl in 30 mL TBST. The solution was added to the membrane and incubated at room temperature for 1 hour with gentle shaking.

The secondary antibody solution was removed and washed 3 times with 50 mL TBST, as described above.

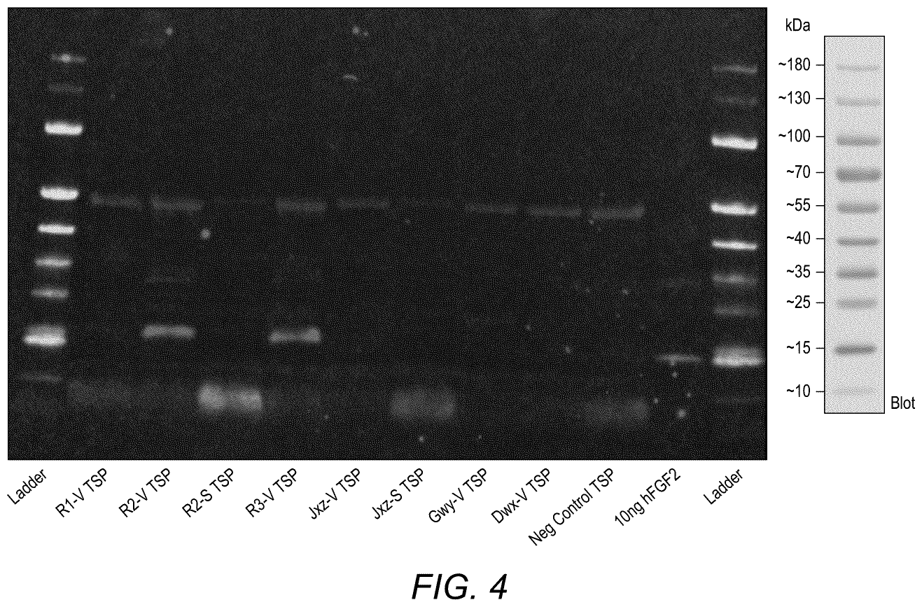

The membrane was imaged at 700 nm (ladder) and 800 nm h-FGF2. Human FGF-2 (SEQ ID NO: 2) is approximately 18 kDa. R2-V TSP (Lane 3) and R3-V TSP (Lane 5) show expression of human FGF-2 (FIG. 4). The expression construct used in Lane 5 is derived from the construct illustrated in FIG. 3 but lacks the TMV Omega enhancer.

Quantification of hFGF-2 was performed using the hu-FGF basic ELISA kit from Invitrogen and performed according to manufacturer's protocol. ELISA was performed on the total soluble protein (TSP). The TSP was tested at two dilutions: 2x and 20x. Calculated yield based on standard curve was 560-808 pg/ml.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 1
Amino Acid sequences of Mammalian Proteins

| SEQ ID NO | Name | Sequence |
|---|---|---|
| SEQ ID NO 1 | Human FGF-2 | MVGVGGGDVEDVTPRPGGCQISGRGARGCNGIPGA |
| | (full length) | AAWEAALPRRRPRRHPSVNPRSRAAGSPRTRGRRTE |
| | | ERPSGSRLGDRGRGRALPGGRLGGRGRGRAPERVG |
| | | GRGRGRGTAAPRAAPAARGSRPGPAGTMAAGSITT |
| | | LPALPEDGGSGAFPPGHFKDPKRLYCKNGGFFLRIHP |
| | | DGRVDGVREKSDPHIKLQLQAEERGVVSIKGVCAN |
| | | RYLAMKEDGRLLASKCVTDECFFFERLESNNYNTY |
| | | RSRKYTSWYVALKRTGQYKLGSKTGPGQKAILFLP |
| | | MSAKS |
| SEQ ID NO 2 | Truncated human | MAAGSITTLPALPEDGGSGAFPPGHFKDPKRLYCKN |
| | FGF-2 (18 kDa) | GGFFLRIHPDGRVDGVREKSDPHIKLQLQAEERGVV |
| | | SIKGVCANRYLAMKEDGRLLASKCVTDECFFFERLE |
| | | SNNYNTYRSRKYTSWYVALKRTGQYKLGSKTGPGQ |
| | | KAILFLPMSAKS |
| SEQ ID NO 3 | >sp\|P09038\|FGF2 | MVGVGGGDVEDVTPRPGGCQISGRGARGCNGIPGA |
| | HUMAN | AAWEAALPRRRPRRHPSVNPRSRAAGSPRTRGRRTE |
| | Fibroblast growth | ERPSGSRLGDRGRGRALPGGRLGGRGRGRAPERVG |
| | factor 2 | GRGRGRGTAAPRAAPAARGSRPGPAGTMAAGSITT |
| | OS = Homo | LPALPEDGGSGAFPPGHFKDPKRLYCKNGGFFLRIHP |
| | sapiens OX = 9606 | DGRVDGVREKSDPHIKLQLQAEERGVVSIKGVCAN |
| | GN = FGF2 PE = 1 | RYLAMKEDGRLLASKCVTDECFFFERLESNNYNTY |
| | SV = 3 | RSRKYTSWYVALKRTGQYKLGSKTGPGQKAILFLP |
| | | MSAKS |
| SEQ ID NO 4 | >sp\|P09038- | MGDRGRGRALPGGRLGGRGRGRAPERVGGRGRGR |
| | 1\|FGF2_HUMAN | GTAAPRAAPAARGSRPGPAGTMAAGSITTLPALPED |
| | Isoform 2 of | GGSGAFPPGHFKDPKRLYCKNGGFFLRIHPDGRVDG |
| | Fibroblast growth | VREKSDPHIKLQLQAEERGVVSIKGVCANRYLAMK |
| | factor 2 | EDGRLLASKCVTDECFFFERLESNNYNTYRSRKYTS |
| | OS = Homo | WYVALKRTGQYKLGSKTGPGQKAILFLPMSAKS |
| | sapiens OX = 9606 | |
| | GN = FGF2 | |
| SEQ ID NO 5 | >sp\|P09038- | MAAGSITTLPALPEDGGSGAFPPGHFKDPKRLYCKN |
| | 2\|FGF2_HUMAN | GGFFLRIHPDGRVDGVREKSDPHIKLQLQAEERGVV |
| | Isoform 3 of | SIKGVCANRYLAMKEDGRLLASKCVTDECFFFERLE |
| | Fibroblast growth | SNNYNTYRSRKYTSWYVALKRTGQYKLGSKTGPGQ |
| | factor 2 | KAILFLPMSAKS |
| | OS = Homo | |
| | sapiens OX = 9606 | |
| | GN = FGF2 | |
| SEQ ID NO 6 | >sp\|P09038- | MGGRGRGRAPERVGGRGRGRGTAAPRAAPAARGS |
| | 3\|FGF2_HUMAN | RPGPAGTMAAGSITTLPALPEDGGSGAFPPGHFKDP |
| | Isoform 4 of | KRLYCKNGGFFLRIHPDGRVDGVREKSDPHIKLQLQ |
| | Fibroblast growth | AEERGVVSIKGVCANRYLAMKEDGRLLASKCVTDE |
| | factor 2 | CFFFERLESNNYNTYRSRKYTSWYVALKRTGQYKL |
| | OS = Homo | GSKTGPGQKAILFLPMSAKS |
| | sapiens OX = 9606 | |
| | GN = FGF2 | |
| SEQ ID NO 7 | SICYS8 | AHLEYVENLNVKEQLVAGTLYYITLVATDAGKKKI |
| | | YETKIWVKEWEDFKKVVEFKLVGDDSPNPGGITNV |
| | | PFPNLPQFKDLARFAVQDYNKKENAHLEFVENLNV |
| | | KEQVVAGIIYYITLVATDAGKKKIYETKILVKGWEN |
| | | FKEVQEFKLVGDATK |
| SEQ ID NO 8 | Chicken FGF-2 | MAAGAAGSITTLPALPDDGGGGAFPPGHFKDPKRLY |
| | (NP_990764.1) | CKNGGFFLRINPDGRVDGVREKSDPHIKLQLQAEER |
| | | GVVSIKGVSANRFLAMKEDGRLLALKCATEECFFFE |
| | | RLESNNYNTYRSRKYSDWYVALKRTGQYKPGPKTG |
| | | PGQKAILFLPMSAKS |
| SEQ ID NO 9 | Human IL-1beta | MHHHHHHHHHHHHDDDDDKAPVRSLNCTLRDSQQK |
| | | SLVMSGPYELKALHLQGQDMEQQVVFSMSFVQGEE |
| | | SNDKIPVALGLKEKNLYLSCVLKDDKPTLQLESVDP |
| | | KNYPKKKMEKRFVFNKIEINNKLEFESAQFPNWYIS |
| | | TSQAENMPVFLGGTKGGQDITDFTMQFVSS |
| SEQ ID NO 10 | TGF-1 beta | ALDTNYCFSSTEKNCCVRQLYIDFRKDLGWKWIHEP |
| | human/bovine/ | KGYHANFCLGPCPYIWSLDTQYSKVLALYNQHNPG |
| | porcine | ASAAPCCVPQALEPLPIVYYVGRKPKVEQLSNMIVR |
| | Active form | SCKCS |
| SEQ ID NO 11 | TGF-1 beta | DLDTDYCFGPGTDEKNCCVRPLYIDFRKDLQW |
| | Chicken | KWIHEPKGYMANFCMGPCPYIWSADTQYTKVLALY |
| | Active form | NQHNPGASAAPCCVPQTLDPLPIIYYVGRNVRVEQL |
| | | SNMVVRACKCS |
| SEQ ID NO 12 | IGF-1 | GPETLCGAELVDALQFVCGDRGFYFNKPTG |
| | human/bovine/ | YGSSSRRAPQTGIVDECCFRSCDLRRLEMY |
| | porcine | CAPLKPAKSA |
| | Active form | |
| SEQ ID NO 13 | IGF-1 Chicken | GPETLCGAELVDALQFVCGDRGFYFSKPTG |
| | Active form | YGSSSRRLHHKGIVDECCFQSCDLRRLEMY |
| | | CAPIKPPKSA |
| SEQ ID NO 14 | IGF-2 human | MGIPMGKSMLVLLTFLAFASCCIAAYRPSE |
| | Mature form | TLCGGELVDTLQFVCGDRGFYFSRPASRVS |
| | | RRSRGIVEECCFRSCDLALLETYCATPAKS |
| | | ERDVSTPPTVLPDNFPRYPVGKFFQYDTWK |
| | | QSTQRLRRGLPALLRARRGHVLAKELEAFR |
| | | EAKRHRPLIALPTQDPAHGGAPPEMASNRK |
| SEQ ID NO 15 | IGF-2 bovine | MGITAGKSVLVLLAFLAFASCCYAAYRPSE |
| | Mature form | TLCGGELVDTLQFVCGDRGFYFSRPSSRIN |
| | | RRSRGIVEECCFRSCDLALLETYCATPAKS |
| | | ERDVSASTTVLPDDVTAYPVGKFFQYDIWK |
| | | QSTQRLRRGLPAFLRARRGRTLAKELEALR |
| | | EAKSHRPLIALPTQDPATHGGASSKASSD |
| SEQ ID NO 16 | IGF-2 porcine | MGIPMRKPLLVLLVFLALASCCYAAYRPSETLCGGE |
| | Mature form | LVDTLQFVCGDRGFYFSRPASRVNRRSRGIVEECCF |
| | | RSCDLALLETYCATPAKSERDVSTPPTVLPDNFPRYP |
| | | VGKFFRYDTWKQSAQRLRRGLPALLRARRGRTLAK |
| | | ELEAVREAKRHRPLTARPTRDPAAHGGASPEASGHR |
| | | K |
| SEQ ID NO 17 | IGF-2 chicken | MCAARQILLLLLAFLAYALDSAAAYGTAETLCGGEL |
| | Mature form | VDTLQFVCGDRGFYFSRPVGRNNRRINRGIVEECCF |
| | | RSCDLALLETYCAKSVKSERDLSATSLAGLPALNKE |
| | | SFQKPSHAKYSKYNVWQKKSSQRLQREVPGILRAR |
| | | RYRWQAEGLQAAEEARAMHRPLISLPSQRPPAPRAS |
| | | PEATGPQE |
| SEQ ID NO 18 | Activin A human | MPLLWLRGFLLASCWIIVRSSPTPGSEGHSAAPDCPS |
| | | CALAALPKDVPNSQPEMVEAVKKHILNMLHLKKRP |
| | | DVTQPVPKAALLNAIRKLHVGKVGENGYVEIEDDIG |
| | | RRAEMNELMEQTSEIITFAESGTARKTLHFEISKEGS |
| | | DLSVVERAEVWLFLKVPKANRTRTKVTIRLFQQQK |
| | | HPQGSLDTGEEAEEVGLKGERSELLLSEKVVDARKS |
| | | TWHVFPVSSSIQRLLDQGKSSLDVRIACEQCQESGAS |
| | | LVLLGKKKKKEEEGEGKKKGGGEGGAGADEEKEQ |
| | | SHRPFLMLQARQSEDHPHRRRRRGLECDGKVNICCK |
| | | KQFFVSFKDIGWNDWIIAPSGYHANYCEGECPSHIA |
| | | GTSGSSLSFHSTVINHYRMRGHSPFANLKSCCVPTKL |
| | | RPMSMLYYDDGQNIIKKDIQNMIVEECGCS |
| SEQ ID NO 19 | BMP-4 human | MIPGNRMLMVVLLCQVLLGGASHASLIPETGKKKV |
| | | AEIQGHAGGRRSGQSHELLRDFEATLLQMFGLRRRP |
| | | QPSKSAVIPDYMRDLYRLQSGEEEEEQIHSTGLEYPE |
| | | RPASRANTVRSFHHEEHLENIPGTSENSAFRFLFNLSS |
| | | IPENEVISSAELRLFREQVDQGPDWERGFHRINIYEV |
| | | MKPPAEVVPGHLITRLLDTRLVHHNVTRWETFDVSP |
| | | AVLRWTREKQPNYGLAIEVTHLHQTRTHQGQHVRI |
| | | SRSLPQGSGNWAQLRPLLVTFGHDGRGHALTRRRR |
| | | AKRSPKHHSQRARKKNKNCRRHSLYVDFSDVGWN |
| | | DWIVAPPGYQAFYCHGDCPFPLADHLNSTNHAIVQT |
| | | LVNSVNSSIPKACCVPTELSAISMLYLDEYDKVVLK |
| | | NYQEMVVEGCGCR |
| SEQ ID NO 20 | VEGF-A | MTDRQTDTAPSPSYHLLPGRRRTVDAAASRGQGPEP |
| | | APGGGVEGVGARGVALKLFVQLLGCSRFGGAVVRA |
| | | GEAEPSGAARSASSGREEPQPEEGEEEEEKEEERGPQ |
| | | WRLGARKPGSWTGEAAVCADSAPAARAPQALARA |
| | | SGRGGRVARRGAEESGPPHSPSRRGSASRAGPGRAS |
| | | ETMNFLLSWVHWSLALLLYLHHAKWSQAAPMAEG |
| | | GGQNHHEVVKFMDVYQRSYCHPIETLVDIFQEYPDE |
| | | IEYIFKPSCVPLMRCGGCCNDEGLECVPTEESNITMQ |
| | | IMRIKPHQGQHIGEMSFLQHNKCECRPKKDRARQEK |
| | | KSVRGKGKG |
| | | QKRKRKKSRYKSWSVYVGARCCLMPWSLPGPHPC |
| | | GPCSERRKHLFVQDPQTCKCSCKNTDSRCKARQLEL |
| | | NERTCRCDKPRR |

TABLE 2
Nucleotide Sequences of Mammalian Proteins

| | | |
|---|---|---|
| SEQ ID NO 21 | Chicken FGF-2 | GAGGCTGGACGGCCGCGGCAGGGGGCGAGCCCGC |
| | | CCGGCGCTGGCGGCGGCGGCCGGCGGGGGCCCGG |
| | | GGCGGCGGGGAGCCGCCGGGGCCCGGCGCATGGC |
| | | GGCGGGGGCGGCGGGGAGCATCACCACGCTGCCG |
| | | GCGCTGCCCGACGACGGGGGCGGCGGCGCTTTTC |
| | | CCCCCGGGCACTTCAAGGACCCCAAGCGGCTCTA |
| | | CTGCAAGAACGGCGGCTTCTTCCTGCGCATCAACC |
| | | CCGACGGCAGGGTGGACGGCGTCCGCGAGAAGAG |
| | | CGATCCGCACATCAAACTGCAGCTTCAAGCAGAA |
| | | GAAAGAGGAGTAGTATCAATCAAAGGCGTAAGTG |
| | | CAAACCGCTTTCTGGCTATGAAGGAGGATGGCAG |
| | | ATTGCTGGCACTGAAATGTGCAACAGAGGAATGT |
| | | TTCTTTTTCGAGCGCTTGGAATCTAATAACTATAA |
| | | CACTTACCGGTCACGGAAGTACTCTGATTGGTATG |
| | | TGGCACTGAAAAGGACTGGACAGTACAAGCCCGG |
| | | ACCAAAAACTGGACCTGGACAGAAAGCTATCCTT |
| | | TTTCTTCCAATGTCTGCTAAAAGCTGA |
| SEQ ID NO 22 | SICYS8 | ATGGCGCATCTGGAATATGTGGAAAACCTGAACG |
| | | TGAAAGAACAGCTGGTGGCGGGCACCCTGTATTA |
| | | TATTACCCTGGTGGCGACCGATGCGGGCAAAAAA |
| | | AAAATTTATGAAACCAAAATTTGGGTGAAAGAAT |
| | | GGGAAGATTTTAAAAAAGTGGTGGAATTTAAACT |
| | | GGTGGGCGATGATAGCCCGAACCCGGGCGGCATT |
| | | ACCAACGTGCCGTTTCCGAACCTGCCGCAGTTTAA |
| | | AGATCTGGCGCGCTTTGCGGTGCAGGATTATAAC |
| | | AAAAAAGAAAACGCGCATCTGGAATTTGTGGAAA |
| | | ACCTGAACGTGAAAGAACAGGTGGTGGCGGGCAT |
| | | TATTTATTATATTACCCTGGTGGCGACCGATGCGG |
| | | GCAAAAAAAAAATTTATGAAACCAAAATTCTGGT |
| | | GAAAGGCTGGGAAAACTTTAAAGAAGTGCAGGAA |
| | | TTTAAACTGGTGGGCGATGCGACCAAATAG |
| SEQ ID NO 23 | Human FGF-2 | CTGGTGGGTGTGGGGGGTGGAGATGTAGAAGATG |
| | (full length) | TGACGCCGCGGCCCGGCGGGTGCCAGATTAGCGG |
| | | ACGCGGTGCCCGCGGTTGCAACGGGATCCCGGGC |
| | | GCTGCAGCTTGGGAGGCGGCTCTCCCCAGGCGGC |
| | | GTCCGCGGAGACACCCATCCGTGAACCCCAGGTC |
| | | CCGGGCCGCCGGCTCGCCGCGCACCAGGGGCCGG |
| | | CGGACAGAAGAGCGGCCGAGCGGCTCGAGGCTGG |
| | | GGGACCGCGGGCGCGGCCGCGCGCTGCCGGGCGG |
| | | GAGGCTGGGGGGCCGGGGCCGGGGCCGTGCCCCG |
| | | GAGCGGGTCGGAGGCCGGGGCCGGGGCCGGGGG |
| | | ACGGCGGCTCCCCGCGCGGCTCCAGCGGCTCGGG |
| | | GATCCCGGCCGGGCCCCGCAGGGACCATGGCAGC |
| | | CGGGAGCATCACCACGCTGCCCGCCTTGCCCGAG |
| | | GATGGCGGCAGCGGCGCCTTCCCGCCCGGCCACT |
| | | TCAAGGACCCCAAGCGGCTGTACTGCAAAAACGG |
| | | GGGCTTCTTCCTGCGCATCCACCCCGACGGCCGAG |
| | | TTGACGGGGTCCGGGAGAAGAGCGACCCTCACAT |
| | | CAAGCTACAACTTCAAGCAGAAGAGAGAGGAGTT |
| | | GTGTCTATCAAAGGAGTGTGTGCTAACCGTTACCT |
| | | GGCTATGAAGGAAGATGGAAGATTACTGGCTTCT |
| | | AAATGTGTTACGGATGAGTGTTTCTTTTTTGAACG |
| | | ATTGGAATCTAATAACTACAATACTTACCGGTCAA |
| | | GGAAATACACCAGTTGGTATGTGGCACTGAAACG |
| | | AACTGGGCAGTATAAACTTGGATCCAAAACAGGA |
| | | CCTGGGCAGAAAGCTATACTTTTTCTTCCAATGTC |
| | | TGCTAAGAGCTGA |
| SEQ ID NO 24 | Truncated human | CGCGTGGATGGCGTGCGCGAAAAAAGCGATCCGC |
| | FGF-2 (18 kDa) | ATATTAAACTGCAGCTGCAGGCGGAAGAACGCGG |
| | | CGTGGTGAGCATTAAAGGCGTGTGCGCGAACCGC |
| | | TATCTGGCGATGAAAGAAGATGGCCGCCTGCTGG |
| | | CGAGCAAATGCGTGACCGATGAATGCTTTTTTTTT |
| | | GAACGCCTGGAAAGCAACAACTATAACACCTATC |
| | | GCAGCCGCAAATATACCAGCTGGTATGTGGCGCT |
| | | GAAACGCACCGGCCAGTATAAACTGGGCAGCAAA |
| | | ACCGGCCCGGGCCAGAAAGCGATTCTGTTTCTGCC |
| | | GATGAGCGCGAAAAGC |
| SEQ ID NO 25 | >sp\|P09038\|FGF2 | CTGGTGGGTGTGGGGGGTGGAGATGTAGAAGATG |
| | HUMAN | TGACGCCGCGGCCCGGCGGGTGCCAGATTAGCGG |
| | Fibroblast growth | ACGCGGTGCCCGCGGTTGCAACGGGATCCCGGGC |
| | factor 2 | GCTGCAGCTTGGGAGGCGGCTCTCCCCAGGCGGC |
| | OS = Homo | GTCCGCGGAGACACCCATCCGTGAACCCCAGGTC |
| | sapiens OX = 9606 | CCGGGCCGCCGGCTCGCCGCGCACCAGGGGCCGG |
| | GN = FGF2 PE = 1 | CGGACAGAAGAGCGGCCGAGCGGCTCGAGGCTGG |
| | SV = 3 | GGGACCGCGGGCGCGGCCGCGCGCTGCCGGGCGG |
| | | GAGGCTGGGGGGCCGGGGCCGGGGCCGTGCCCCG |
| | | GAGCGGGTCGGAGGCCGGGGCCGGGGCCGGGGG |
| | | ACGGCGGCTCCCCGCGCGGCTCCAGCGGCTCGGG |
| | | GATCCCGGCCGGGCCCCGCAGGGACCATGGCAGC |
| | | CGGGAGCATCACCACGCTGCCCGCCTTGCCCGAG |
| | | GATGGCGGCAGCGGCGCCTTCCCGCCCGGCCACT |
| | | TCAAGGACCCCAAGCGGCTGTACTGCAAAAACGG |
| | | GGGCTTCTTCCTGCGCATCCACCCCGACGGCCGAG |
| | | TTGACGGGGTCCGGGAGAAGAGCGACCCTCACAT |
| | | CAAGCTACAACTTCAAGCAGAAGAGAGAGGAGTT |
| | | GTGTCTATCAAAGGAGTGTGTGCTAACCGTTACCT |
| | | GGCTATGAAGGAAGATGGAAGATTACTGGCTTCT |
| | | AAATGTGTTACGGATGAGTGTTTCTTTTTTGAACG |
| | | ATTGGAATCTAATAACTACAATACTTACCGGTCAA |
| | | GGAAATACACCAGTTGGTATGTGGCACTGAAACG |
| | | AACTGGGCAGTATAAACTTGGATCCAAAACAGGA |
| | | CCTGGGCAGAAAGCTATACTTTTTCTTCCAATGTC |
| | | TGCTAAGAGCTGA |
| SEQ ID NO 26 | >sp\|P09038-1 | ATGGCAGCCGGGAGCATCACCACGCTGCCCGCCT |
| | \|FGF2_HUMAN | TGCCCGAGGATGGCGGCAGCGGCGCCTTCCCGCC |
| | Isoform 2 of | CGGCCACTTCAAGGACCCCAAGCGGCTGTACTGC |
| | Fibroblast growth | AAAAACGGGGGCTTCTTCCTGCGCATCCACCCCG |
| | factor 2 | ACGGCCGAGTTGACGGGGTCCGGGAGAAGAGCGA |
| | OS = Homo | CCCTCACATCAAGCTACAACTTCAAGCAGAAGAG |
| | sapiens OX = 9606 | AGAGGAGTTGTGTCTATCAAAGGAGTGTGTGCTA |
| | GN = FGF2 | ACCGTTACCTGGCTATGAAGGAAGATGGAAGATT |
| | | ACTGGCTTCTAAATGTGTTACGGATGAGTGTTTCT |
| | | TTTTTGAACGATTGGAATCTAATAACTACAATACT |
| | | TACCGGTCAAGGAAATACACCAGTTGGTATGTGG |
| | | CACTGAAACGAACTGGGCAGTATAAACTTGGATC |
| | | CAAAACAGGACCTGGGCAGAAAGCTATACTTTTT |
| | | CTTCCAATGTCTGCTAAGAGCTGA |
| SEQ ID NO 27 | IL 1-beta | CTCGAGATGCATCATCATCATCATCATCATCATCA |
| | | TCATCATCATGATGATGATGATAAAGCACCTGTAC |
| | | GATCACTGAACTGCACGCTCCGGGACTCACAGCA |
| | | AAAAAGCTTGGTGATGTCTGGTCCATATGAACTG |
| | | AAAGCTCTCCACCTCCAGGGACAGGATATGGAGC |
| | | AACAAGTGGTGTTCTCCATGTCCTTTGTACAAGGA |
| | | GAAGAAAGTAATGACAAAATACCTGTGGCCTTGG |
| | | GCCTCAAGGAAAAGAATCTGTACCTGTCCTGCGT |
| | | GTTGAAAGATGATAAGCCCACTCTACAGCTGGAG |
| | | AGTGTAGATCCCAAAAATTACCCAAAGAAGAAGA |
| | | TGGAAAAGCGATTTGTCTTCAACAAGATAGAAAT |
| | | CAATAACAAGCTGGAATTTGAGTCTGCCCAGTTCC |
| | | CCAACTGGTACATCAGCACCTCTCAAGCAGAAAA |
| | | CATGCCCGTCTTCCTGGGAGGGACCAAAGGCGGC |
| | | CAGGATATAACTGACTTCACCATGCAATTTGTGTC |
| | | TTCCTAAACGCGT |
| SEQ ID NO 28 | FGF-2 stable | CGCGTGGATGGCGTGCGCGAAAAAAGCGATCCGC |
| | mutant (human) | ATATTAAACTGCAGCTGCAGGCGGAAGAACGCGG |
| | | CGTGGTGAGCATTAAAGGCGTGTGCGCGAACCGC |
| | | TATCTGGCGATGAAAGAAGATGGCCGCCTGCTGG |
| | | CGAGCAAATGCGTGACCGATGAATGCTTTTTTTTT |
| | | GAACGCCTGGAAAGCAACAACTATAACACCTATC |
| | | GCAGCCGCAAATATACCAGCTGGTATGTGGCGCT |
| | | GAAACGCACCGGCCAGTATAAACTGGGCAGCAAA |
| | | ACCGGCCCGGGCCAGAAAGCGATTCTGTTTCTGCC |
| | | GATGAGCGCGAAAAGC |

1-77. (canceled)

78. A plant cell, wherein the plant cell comprises:
a vector comprising:
(i) a polynucleotide sequence encoding for a heterologous protease inhibitor or a functional variant thereof; and
(ii) a polynucleotide sequence encoding for a mammalian protein or a functional variant thereof,
wherein the mammalian protein is a mammalian growth factor protein selected from the group consisting of mammalian fibroblast growth factor 2 (FGF-2), mammalian insulin-like growth factor 1 (IGF-1), and mammalian vascular endothelial growth factor (VEGF).

79. The plant cell of claim 78, wherein the heterologous protease inhibitor is SICYS8.

80. The plant cell of claim 78, wherein the vector is:
(i) a viral vector; or
(ii) a bacterial vector.

81. The plant cell of claim 78, wherein the vector is Tobacco mosaic virus.

82. The plant cell of claim 78, wherein the vector is an Agrobacterium species.

83. The plant cell of claim 78, wherein the plant cell is a tobacco plant cell.

84. The plant cell of claim 83, wherein the tobacco plant cell is an Nicotiana benthamiana cell.

85. The plant cell of claim 78, wherein the mammalian protein is the mammalian FGF-2, and wherein the mammalian FGF-2 is human FGF-2 or bovine FGF-2.

86. The plant cell of claim 85, wherein the polynucleotide sequence encoding for the human FGF-2 comprises a sequence with at least 75% sequence identity to SEQ ID NOs: 23, 24, 25, 26, or 28.

87. The plant cell of claim 85, wherein the human FGF-2 comprises a polypeptide sequence selected from the group consisting of SEQ ID NOs: 1, 2, 3, 4, 5, 6, and 14.

88. The plant cell of claim 85, wherein the bovine FGF-2 comprises a polypeptide sequence of SEQ ID NO: 15.

89. The plant cell of claim 78, wherein the mammalian protein is the mammalian IGF-1.

90. The plant cell of claim 12, wherein the mammalian IGF-1 comprises a polypeptide sequence of SEQ ID NO: 12 or SEQ ID NO: 13.

91. The plant cell of claim 78, wherein the mammalian protein is the mammalian VEGF.

92. The plant cell of claim 78, wherein the mammalian VEGF comprises a polypeptide sequence of SEQ ID NO: 20.

93. A method of manufacturing a mammalian protein, comprising:
(i) culturing a plurality of plant cells, each of the plurality of plant cells comprising a vector comprising:
(a) a polynucleotide sequence encoding for a heterologous protease inhibitor or a functional variant thereof, and
(b) a polynucleotide sequence encoding for a mammalian protein or a functional variant thereof; and
(ii) extracting soluble proteins from a harvested plurality of plant cells, thereby generating an extraction product comprising the mammalian protein or a functional variant thereof,
wherein the mammalian protein is a mammalian growth factor protein selected from the group consisting of mammalian fibroblast growth factor 2 (FGF-2), mammalian insulin-like growth factor 1 (IGF-1), and mammalian vascular endothelial growth factor (VEGF).

94. The method of claim 93, further comprising: prior to and/or during (i), infiltrating the vector into another plurality of plant cells, thereby forming the plurality of plant cells.

95. The method of claim 94, wherein the infiltrating is vacuum infiltrating.

96. The method of claim 93, wherein the mammalian protein is human FGF-2, bovine FGF-2, bovine IGF-1, or human VEGF.

97. The method of claim 93, wherein the mammalian protein is histidine-tagged, and wherein the extracting comprises using metal-based histidine-tag affinity (NTA) magnetic beads.
